(19) United States
(12) Patent Application Publication
(10) Pub. No.: US 2006/0169281 A1
(43) Pub. Date: Aug. 3, 2006

(54) Continuous flow selective delivery of therapeutic gas

(76) Inventors: Alonzo C. Aylsworth, Wildwood, MO (US); Charles R. Aylsworth, Wildwood, MO (US); Lawrence C. Spector, Austin, TX (US)

Correspondence Address: CONLEY ROSE, P.C., P. O. BOX 3267, HOUSTON, TX, 77253-3267, US

(21) Appl. No.: 11/344,646

(22) Filed: Feb. 1, 2006

Related U.S. Application Data
(60) Provisional application No. 60/649,507, filed on Feb. 3, 2005.

(51) Int. Cl. A61M 16/00; A62B 7/04

(52) U.S. Cl. 128/204.23; 128/204.21; 128/204.18; 128/204.26

(57) ABSTRACT

A method and system of continuous flow selective delivery. At least some of the illustrative embodiments are methods comprising sensing an attribute of respiratory airflow of a first breathing orifice of a patient, and delivering a continuous flow of therapeutic gas to a second breathing orifice of the patient simultaneously with the sensing.

### CROSS-REFERENCE TO RELATED APPLICATIONS

This specification claims the benefit of Provisional Application Ser. No. 60/649,507, filed Feb. 3, 2005, titled “Continuous Flow Selective Delivery of Therapeutic Gas,” which application is incorporated by reference herein as if reproduced in full below.

### STATEMENT REGARDING FEDERALLY SPONSORED RESEARCH OR DEVELOPMENT

Not applicable.

### BACKGROUND

Patients with respiratory ailments may be required to breathe a therapeutic gas, such as oxygen. The therapeutic gas may be delivered to the patient from a therapeutic gas source by way of a nasal cannula. Delivery of therapeutic gas to a patient may be continuous or in a conserve mode. In continuous delivery, the therapeutic gas may be supplied at a constant flow throughout the patient's breathing cycle. If the patient has a blocked naris, however (e.g. because of congestion or a physical abnormality), the therapeutic gas delivered to that naris is wasted, and also the patient's blood oxygen saturation may drop to the point of desaturation. Moreover, if the nasal cannula becomes dislodged, such as during sleep, the therapeutic gas continuously delivered to a nasal prong that is not in operational relationship to a naris is wasted.

### SUMMARY

The problems noted above are solved in large part by a method and system of continuous flow selective delivery. At least some of the illustrative embodiments are methods comprising sensing an attribute of respiratory airflow of a first breathing orifice of a patient, and delivering a continuous flow of therapeutic gas to a second breathing orifice of the patient simultaneously with the sensing.

Other illustrative embodiments are methods comprising delivering therapeutic gas to one or more breathing orifices of a patient, and then ceasing delivery of therapeutic gas for a predetermined number of respirations and sensing an attribute of airflow through each breathing orifice during the ceasing, and thereafter delivering a continuous flow of therapeutic gas one of: substantially only to the breathing orifice exhibiting greater airflow; or to each breathing orifice.

Other illustrative embodiments are systems comprising a processor, a first sensor electrically coupled to the processor and configured to fluidly couple to a breathing orifice of a patient (the sensor senses an attribute of airflow of the breathing orifice), and a first valve electrically coupled to the processor and configured to selectively fluidly couple a source of therapeutic gas to a breathing orifice. The system is configured to sense the attribute of airflow of a first breathing orifice, sense the attribute of airflow of a second breathing orifice, and based at least in part on the attributes of airflow sensed, one of: deliver a continuous flow of therapeutic gas to only the first breathing orifice; deliver the continuous flow of therapeutic gas to only a second breathing orifice; or deliver the continuous flow of therapeutic gas to each of the first and second breathing orifices.

Yet still other illustrative embodiments are a cannula comprising a first nasal tubing having a device end and an aperture end (wherein the cannula is configured to place the aperture end in fluid communication with a first naris of a patient), a second nasal tubing having a device end and an aperture end (wherein the cannula is configured to place the aperture end of the second nasal tubing in fluid communication with a second naris of the patient), and an oral tubing having a device end and a first and second aperture ends and the oral tubing mechanically coupled to at least one of the first or second nasal tubing (wherein the cannula is configured to place the aperture ends of the oral tubing in fluid communication with a mouth of the patient). The first nasal tubing, the second nasal tubing and the oral tubing are fluidly independent between their aperture ends and their device ends.

The disclosed devices and methods comprise a combination of features and advantages which enable it to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of various embodiments of the invention, reference will now be made to the accompanying drawings in which:

FIG. 1 illustrates a continuous flow selective delivery system in accordance with at least some embodiments of the invention;

FIG. 2A illustrates, in shorthand notation, the system of FIG. 1;

FIG. 2B illustrates alternative embodiments of the system of FIG. 1;

FIG. 2C illustrates alternative embodiments of the system of FIG. 1;

FIG. 2D illustrates alternative embodiments of the system of FIG. 1;

FIG. 2E illustrates alternative embodiments of the system of FIG. 1;

FIG. 2F illustrates alternative embodiments of the system of FIG. 1;

FIG. 3 illustrates a flow diagram of a method that may be implemented in accordance with embodiments of the invention;

FIG. 4 illustrates an additional/or alternative method that may be implemented in accordance with embodiments of the invention;

FIG. 5 illustrates a continuous flow selective delivery system in accordance with alternative embodiments of the invention;

FIG. 6A illustrates, in shorthand notation, the embodiments of FIG. 5;

FIG. 6B illustrates alternative embodiments of the system of FIG. 5;

FIG. 6C illustrates alternative embodiments of the system of FIG. 5;

FIG. 6D illustrates a portion of alternative embodiments of the system of FIG. 5;

FIG. 6E illustrates a portion of alternative embodiments of the system of FIG. 5;

FIG. 7A illustrates a respiratory waveform;

FIG. 7B illustrates a respiratory waveform measured during continuous flow delivery of therapeutic gas; and

FIG. 8 illustrates a method in accordance with alternative embodiments.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

“Continuous flow of therapeutic gas” refers to a therapeutic gas delivery mode in which therapeutic gas is provided to a patient at a substantially constant flow rate throughout both the inhalation and exhalation phase of a patient's respiratory cycle. Continuous flow delivery is in contrast to “bolus” delivery where a bolus of gas is delivered substantially only during the inhalation phase.

“Breathing orifice” refers to the nose, mouth, and/or the nares of the nose individually.

### DETAILED DESCRIPTION

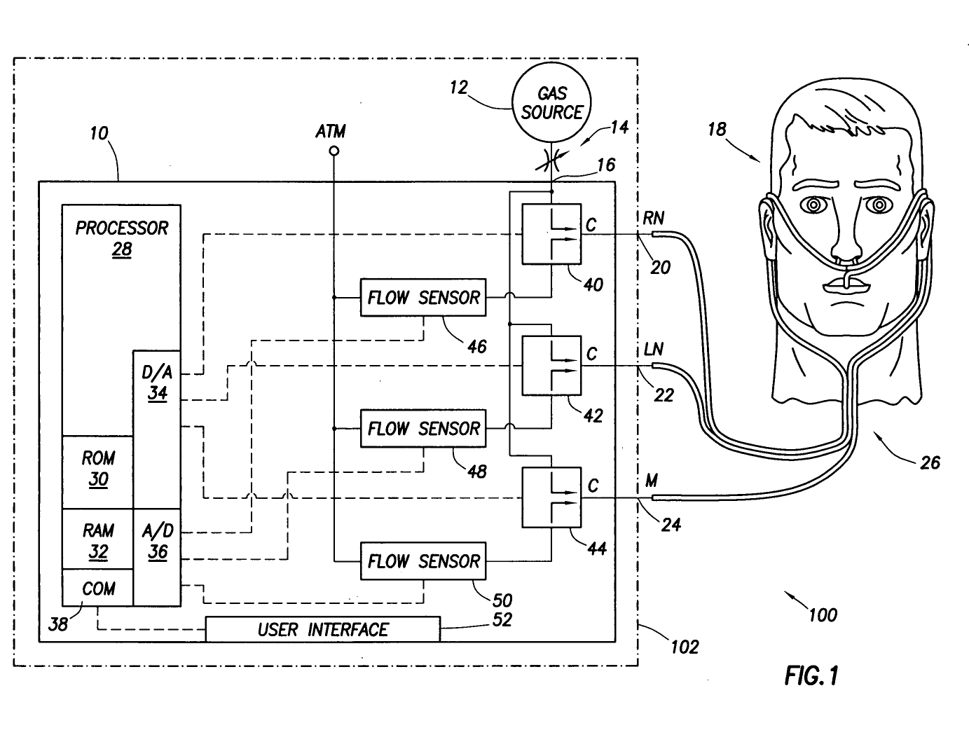

FIG. 1 illustrates a continuous flow selective delivery system 100 in accordance with at least some embodiments of the invention. In particular, a monitoring and control system 10 may be coupled to a therapeutic gas source 12 by way of a flow control device 14 and gas port 16. The therapeutic gas source 12 may be any suitable source of therapeutic gas, such as a portable cylinder of oxygen or nitrous oxide, an oxygen concentration system, a liquid oxygen system, or a permanent supply of therapeutic gas such as in a hospital. In alternative embodiments, the monitoring and control system 10, gas source 12 and flow control device 14 may be an integral system 102. The monitoring and control system 10 couples to a patient 18 by a variety of ports, such as narial ports 20 and 22 and an oral port 24. For example, the monitoring and control system 10 may couple to the patient's nares and mouth by way of a cannula 26. In accordance with embodiments illustrated in FIG. 1, the cannula 26 may have three fluidly independent flow pathways, one each to each of the patient's left naris, right naris and mouth.

In accordance with embodiments of the invention, the monitoring and control system 10 monitors patient breathing through each breathing orifice and selectively delivers therapeutic gas to a left naris (LN), right naris (RN) and/or to the mouth (M) of the patient. More particularly, in some embodiments the monitoring and control system 10 periodically ceases continuous flow delivery to a particular breathing orifice and measures an attribute of airflow of the particular breathing orifice (e.g., some or all of the actual airflow, a pressure associated with the airflow, a temperature associated with the airflow). The remainder of this specification refers to measuring an attribute of airflow as “measuring airflow” or “sensing airflow” because the preferred embodiments use mass flow sensors (discussed below); however, measuring any attribute of airflow is within the contemplation of this specification. The process of sensing airflow through a particular breathing orifice while delivering continuous flow therapeutic gas to one or more other breathing orifices repeats until airflow through each breathing orifice is measured. After the airflow through each breathing orifice is measured, the monitoring and control system 10 then selectively provides a continuous flow of therapeutic gas to the breathing orifice carrying at least a first predetermined threshold of the overall measured airflow, or if two (or three) of the breathing orifices each carry at least the predetermined threshold of the overall measured airflow, the patient's entire therapeutic gas flow prescription is divided among the breathing orifices. Thus, in these embodiments the continuous flow of therapeutic gas to the patient as a whole is not interrupted during the testing phase.

The monitoring and control system 10 comprises both electrical components and mechanical components. In order to differentiate between electrical connections and mechanical connections, FIG. 1 (and the remaining figures) illustrate electrical connections between components with dashed lines, and fluid connections (e.g. tubing connections between devices), with solid lines. The monitoring and control system 10 in accordance with at least some embodiments of the invention comprises a processor 28. The processor 28 may be a microcontroller, and therefore the microcontroller may be integral with read only memory (ROM) 30, random access memory (RAM) 32, a digital-to-analog converter (D/A) 34, and an analog-to-digital converter (A/D) 36. The processor 28 may further comprise communication logic 38, which allows the system 10 to communicate with external devices, e.g., to transfer stored data about a patient's breathing patterns. Although a microcontroller may be preferred because of the integrated components, in alternative embodiments the processor 28 may be implemented by a stand-alone central processing unit in combination with individual RAM, ROM, communication, D/A and A/D devices.

The ROM 30 stores instructions executable by the processor 28. In particular, the ROM 30 may comprise a software program that in whole or in part implements the various embodiments of the invention discussed herein. The RAM 32 may be the working memory for the processor 28, where data may be temporarily stored and from which instructions may be executed. Processor 28 may couple to other devices within the preferential delivery system by way of A/D converter 36 and D/A converter 34.

Monitoring and control system 10 also comprises three-port valve 40, three-port valve 42, and in some embodiments three-port valve 44. Each of these three-port valves may be a five-volt solenoid operated valve that selectively fluidly couples one of two ports to a common port (labeled as C in the drawings). Three-port valves 40, 42 and 44 may be Humprey Mini-Mizers having part No. D3061, available from the John Henry Foster Co., or equivalents. By selectively applying voltage on a digital output signal line coupled to the three-port valve 40, the processor 28 may: couple gas from the gas source 12 to the common port and therefore to the right naris; and couple the flow sensor 46 to the common port and therefore the right naris. Likewise, the three-port valve 42, under command of the processor 28, may: couple gas from the gas source 12 to the left naris; and couple the flow sensor 48 to the left naris. If the patient's mouth is also monitored, three-port valve 44, under command of the processor 28, may: couple gas from the gas source 12 to the patient's mouth; and couple the flow sensor 50 to the patient's mouth. Flow sensors in accordance with some embodiments of the invention are flow-through type sensors, and thus each flow sensor 46, 48 and 50 fluidly couples to an atmospheric vent (marked ATM in the drawing), thus allowing airflow through the flow sensor for measurement purposes.

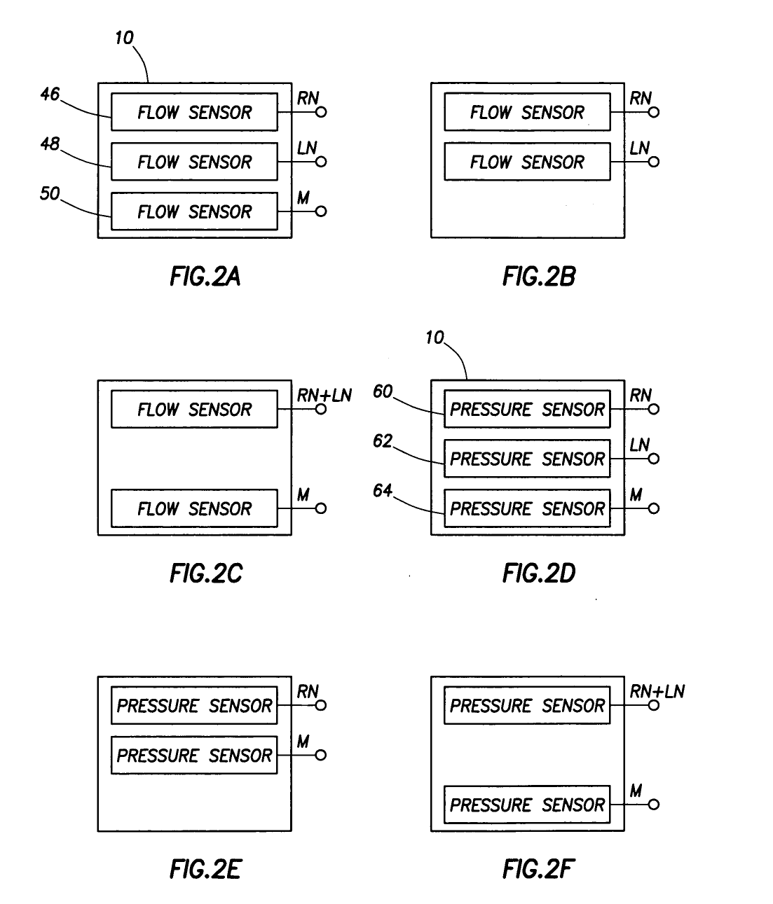

FIG. 2A illustrates the monitoring and control system 10 of FIG. 1 in a shorthand notation, showing only flow sensors 46, 48 and 50. FIG. 2B illustrates alternative embodiments which omit the flow sensor associated with the mouth, and thus these embodiments monitor and deliver therapeutic gas only to the nares of a patient. In the embodiments of FIG. 2B, if both the left naris and right naris are open to flow, the monitoring and control system 10 may deliver therapeutic gas to either naris or to both nares. In the event that either the left or right naris become clogged or blocked such that the airflow falls below a predetermined threshold (e.g. 25% of the sensed airflow), or if the sensing and delivery tubings (such as a nasal cannula) become dislodged, the system may provide therapeutic gas to the naris where airflow is sensed. FIG. 2C illustrates further alternative embodiments where two flow sensors are used, but in this case only one flow sensor is associated with the nares, and the second flow sensor is associated with the mouth. In the embodiments of FIG. 2C, a patient may utilize a single lumen cannula and a sensing and delivery tube associated with the mouth. The monitoring and control system 10 may thus selectively provide therapeutic gas to the nares and/or to the mouth. In the event that either of the nares as a group or the mouth become blocked or otherwise unavailable for inspiration, the monitoring and control system 10 preferably provides therapeutic gas to the breathing orifice through which inhalation takes place.

FIG. 2D illustrates yet further alternative embodiments where, rather than using flow sensors in the monitoring and control system 10, pressure sensors are used. In particular, pressure sensor 60 is configured to fluidly couple to the right naris, pressure sensor 62 is configured to fluidly couple to the left naris, and pressure sensor 64 is configured to fluidly couple to the mouth, such as by use of a cannula 26 (FIG. 1). In these embodiments, the pressure sensors sense pressures indicative of airflow through each breathing orifice. FIG. 2E illustrates alternative embodiments using pressure sensors where only a patient's nares are used for sensing and delivery, and operation of these embodiments is similar to that of FIG. 2B. FIG. 2F illustrates further alternative embodiments where two pressure sensors are used, but in this case only one pressure sensor is associated with the nares, and the second pressure sensor associated with the mouth, and operation of these embodiments is similar to that of FIG. 2C. Other sensors, such as thermal devices (e.g., thermocouples and resistive thermal devices) may be equivalently used, with the temperature sensitive portions placed within the airflow stream, either proximate to the patient or within the system 10.

Consider a situation where the monitoring and control system 10 couples to the nares of the patient by way of a bifurcated nasal cannula with no fluid connection to the mouth of the patient. Further consider that a patient's illustrative 2 liters per minute (LPM) therapeutic gas flow prescription is being simultaneously delivered through each lumen of the bifurcated nasal cannula. With reference to FIG. 1, this illustrative situation occurs when the flow control device 14 is set to allow a 2 LPM flow and the three-port valves 40 and 42 have their common port coupled to the gas source 12 (and assuming the flow sensor 50 and three-port valve 44 are not present or not in use). Assuming the resistance to gas flow through each flow pathway is approximately equal, each lumen or flow pathway of the cannula carries approximately 1 LPM. In accordance with some embodiments of the invention, the monitoring and control system 10 periodically, under command of software executed on processor 28, switches the valve position of three-port valve 40, while leaving the valve position of three-port valve 42 unchanged. Thus, the patient's 2 LPM flow prescription is delivered to the patient's left naris, and the monitoring and control system 10 measures airflow of the patient's right naris. The simultaneous continuous flow delivery to the left naris and airflow detection of the right naris may continue for one or more respiratory cycles, with the processor 28 calculating and storing an indication of the measured airflow and/or measure volume carried by the right naris.

At some point thereafter, monitoring and control system 10 changes the valve position of three-port valves 40 and 42. Thus, the patient's 2 LPM flow prescription is delivered to the patient's right naris, and the monitoring and control system 10 senses airflow of the patient's left naris. The continuous flow delivery to the right naris and simultaneous airflow sensing of the left naris may likewise continue for one or more respiratory cycles, with the processor 28 storing an indication of the measured airflow and/or measured volume carried by the left naris. In embodiments utilizing three-port valve 44 and flow sensor 50, airflow of the patient's mouth may likewise be sensed, and an indication of the measured airflow and/or measured volume recorded (while delivering a continuous flow of therapeutic gas to one or both of the patient's nares).

The processor 28 then makes a determination of the total sensed volume (possibly on a per-breath basis, or an average of all the breaths sensed), and the relative percentage of the volume carried by each breathing orifice. Based on these determinations, the monitoring and control system 10 may: simultaneously deliver therapeutic gas to all three breathing orifices; deliver therapeutic gas only the patient's nares (if the patient's mouth is closed, or if the oral circuit is not utilized); deliver therapeutic gas to only one naris of the patient (because the second naris is congested and thus fully or partially blocked, the second naris is blocked by physical abnormality, or the cannula has slipped off); or deliver therapeutic gas only to the mouth of the patient (because both nares are congested and thus fully or partially blocked, or the cannula has slipped off).

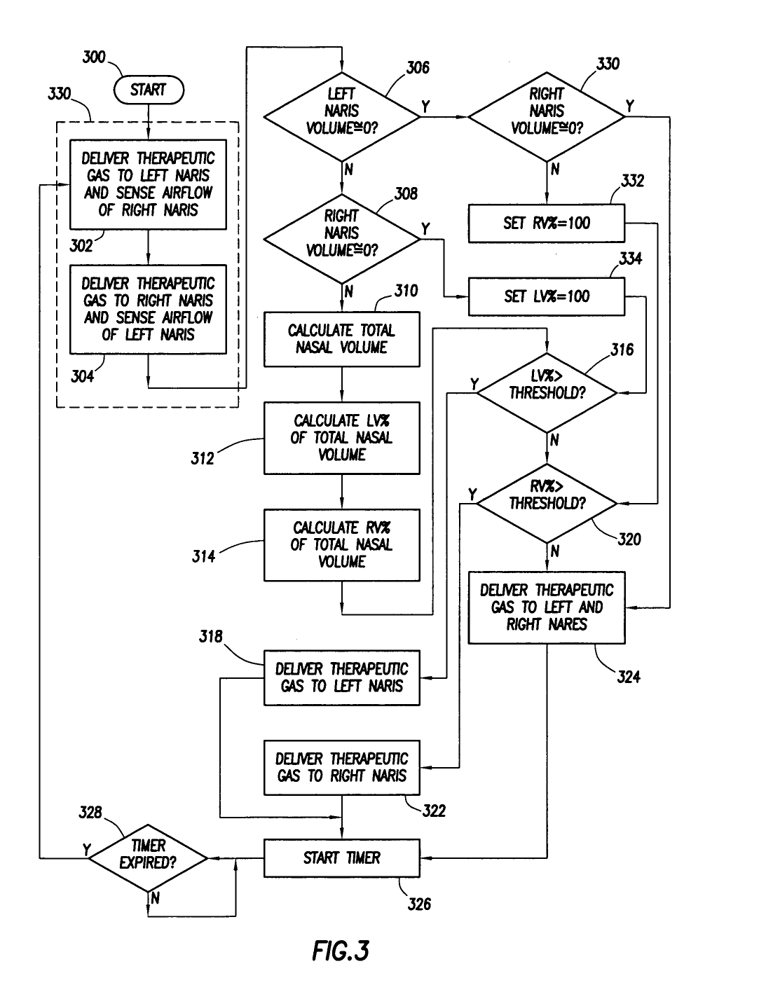

FIG. 3 illustrates a flow diagram of a method that may be implemented in accordance with embodiments of the invention. More particularly, FIG. 3 illustrates sensing airflow of each naris (while delivering a continuous flow of therapeutic gas), and then delivery of therapeutic gas based on measured airflow. The flow diagram of FIG. 3 is with respect to a system coupled to, sensing and delivering only to a patient's nares. Operation of a system that additionally couples to, senses and delivers to a patient's mouth is an extension of illustrative FIG. 3, and is not shown so as not to unduly complicate the figure. Moreover, the method is merely illustrative, and the various steps may be performed in a different order, combined, or some steps omitted, without departing from the scope and spirit of the invention. The process starts (block 300), and moves to delivering therapeutic gas to the left naris while sensing airflow of the right naris (block 302). With brief reference to FIG. 1, this step comprises having three-port valve 42 couple the gas source 12 to its common port, and having three-port valve 40 couple the flow sensor 46 to its common port. Sensing airflow of the left naris while delivering to the right naris make take place for as short a period of time as a single inhalation, or may extend for a plurality of breaths. Returning to FIG. 3, after sensing airflow of the right naris the situation is reversed, and therapeutic gas is delivered to the right naris while sensing airflow of the left naris (block 304). Referring briefly again to FIG. 1, this step comprises having three-port valve 40 couple the gas source 12 to its common port, and having the three-port valve 42 couple flow sensor 48 to its common port.

Still referring to FIG. 3, and skipping for now steps 306 and 308, the next step is calculating the total breath volume, in this case of nasal use only, total nasal volume (block 310). In embodiments using a nasal cannula as the mechanism by which the monitoring and control system 10 (FIG. 1) fluidly couples to the patient 18, the attribute of airflow measured by each sensor will be representative of only a part of the total airflow of the breathing orifice. Thus, the “total volume” calculation (block 310) may be a total sensed volume, possibly created by summing the measured airflows to determine volume for each breathing orifice, then summing the volumes. In alternative embodiments, the monitoring and control system 10 may couple to the patient in such a way that substantially all the airflow of a breathing orifice is monitored. In this specification and in the claims, reference to “total volume” or “total breath volume” refers to total sensed volume in cases where only a portion of the airflow is sensed or where an attribute of airflow is sensed, or the reference may refer to total volume in embodiments where substantially all the airflow is sensed.

After calculating total nasal volume, the next illustrative step is calculating left naris volume percentage (LV %) (block 312), being a percentage carried by the left naris of the total volume. Thereafter, a right naris volume percentage (RV %) is calculated (block 314), being a percentage carried by the right naris of the total volume. If the system operates on the patient's mouth, a mouth volume percentage is calculated as well. The next step in the illustrative method of FIG. 3 is a determination of whether the left naris volume percentage (LV %) is greater than a predetermined threshold (block 316). If so, the monitoring and control system 10 delivers the therapeutic gas only to the left naris (block 318). For example, if the left naris carries 75% or more of the total volume, then therapeutic gas is delivered only to the left naris as delivery of therapeutic gas to the blocked or partially blocked right naris of this illustrative case is most likely wasted, and may result de-saturation of the patient's blood-oxygen. If the left naris volume percentage is less than the predetermined threshold (again block 316), the next step may be a determination of whether the right naris volume percentage is greater than a predetermined threshold (block 320). If so, the monitoring and control system 10 delivers the therapeutic gas only to the right naris (block 322). For example, if the right naris carries 75% or more of the total volume, then therapeutic gas is delivered only to the right naris as delivery of therapeutic gas to the blocked or partially blocked left naris of this illustrative case is most likely wasted, and may result de-saturation of the patient's blood-oxygen. If the right naris volume percentage (RV %) is less than the predetermined threshold (again block 320), then airflow may be roughly evenly divided between the breathing orifices, and thus the monitoring and control system 10 delivers therapeutic gas to both the left and right nares (block 324).

Regardless of whether the continuous flow therapeutic gas is delivered to the left naris only (block 318), the right naris only (block 322), or the both nares (block 324), the next step in the illustrative process may be to start a timer (block 326) and wait for the timer to expire (block 328). In accordance with at least some embodiments, the timer period may be on the order of five minutes. Thus, therapeutic gas is delivered to the selected breathing orifice or orifices while the timer runs. Likewise, the process of determining to which breathing orifice to deliver therapeutic gas may be repeated periodically, with the period set by the timer. After the timer expires (again block 328), the process begins anew by delivering therapeutic gas to the left naris while sensing airflow of the right naris (block 302).

FIG. 3 also illustrates special cases with respect to measured airflow: namely the no airflow (and therefore no carried volume) conditions. In particular, after sensing (blocks 302 and 304), a determination is made as to whether the left naris measured volume is substantially zero (block 306). If the left naris measured volume is substantially zero, a determination is made as to whether the right naris measured volume is substantially zero (block 330). If there is a no flow condition on both nares, either the patient's congestion is such that there is no narial flow, or the cannula has moved from operational relationship with the nose. In either case, a monitoring and control system 10 in accordance with embodiments of the invention attempts to supply therapeutic gas to each naris (block 324) in the hope that at least some of the therapeutic gas finds its way to the patient.

If, on the other hand, the left naris volume is substantially zero (block 306) but right naris measured volume is non-zero (again block 330), then the right naris is the only naris carrying substantial volume. In this case, the patient's prescription of therapeutic gas is delivered to the right naris (regardless of the state of congestion or blockage of the right naris) by assigning right naris volume percentage (RV %) to be 100 percent (block 332), and stepping to the determination of whether the right naris volume percentage is greater than the predetermined threshold (block 320). Given the assignment in this case of right naris volume percentage to be 100%, the method steps to delivery to the right naris (block 322), and the time is started (block 326).

Still referring to FIG. 3, if the situation is reversed, and the left naris measured volume is not zero (block 306) but the right naris measured volume is substantially zero (block 308), then the left naris is the only naris carrying volume. In this case, the patient's prescription of therapeutic gas is delivered to the left naris (regardless of the state of congestion or blockage of the left naris) by assigning the left naris volume percentage (LV %) to be 100 percent (block 334), and stepping to the determination of whether the left naris volume percentage is greater than the predetermined threshold (block 316). Given the assignment in this case of left naris volume percentage to be 100 percent, the method steps to delivering to the left naris (block 318) and the timer is started (block 326).

Thus, the monitoring and control system 10 may beneficially and periodically determine the most appropriate breathing orifice as the patient's state of congestion changes or as the physical causality changes, such as a patient turning to one side causing narial valve collapse. Depending on the chosen predetermined threshold, it is possible that a decision may be made to not deliver to a particular breathing orifice even if some airflow is carried by that breathing orifice. For example, in some embodiments the monitoring and control system may elect not to deliver therapeutic gas to a naris if that naris carries less than 25% of the total volume, even if the carried volume is greater than zero. In this situation, and in the illustrative embodiments of FIG. 1, one of the flow sensors may be coupled to its respective breathing orifice (in order to block therapeutic gas flow) for an extended period of time, such as a timer period (see FIG. 3, blocks 326 and 328). During this period of time, the monitoring and control system 10 may perform other beneficial functions.

In accordance with at least some embodiments of the invention, when a flow sensor (or other sensor) is coupled to a volume carrying breathing orifice, the monitoring and control system 10 monitors the patient for disordered breathing, such as hypopnea, apnea and/or snoring. Apnea is a temporary cessation of breathing, and hypopnea is slow or shallow breathing. A hypopnea event may sometimes precede an apnea event. Though the definition varies from country to country, in the United States the accepted definition of hypopnea is as defined by the American Academy of Sleep Medicine (AASM) in an article titled, “Sleep-Related Breathing Disorders in Adults: Recommendations for Syndrome Definition and Measurement Techniques in Clinical Research” accepted for publication in April 1999 (hereinafter the Chicago Criteria). The Chicago Criteria defines a hypopnea as a “clear decrease (>50%) from baseline in the amplitude of a valid measure of breathing during sleep . . . [and] The event lasts longer than 10 seconds . . . .”Baseline comes in two varieties: “the mean amplitude of stable breathing and oxygenation in the two minutes proceeding onset of the event”; or, “the mean amplitude of the three largest breaths in the two minutes preceding the onset of the event.” Thus, a reduction of measured amplitude by greater than 50% (with a corresponding time factor of 10 seconds) comprises a hypopnea event. Both hypopnea and apnea events may result in lowering of a patient's blood-oxygen saturation to the point where, during sleep, the patient experiences brain arousal which adversely affect sleep. Snoring may be high frequency (relative to breathing) sound caused by vibrations of the soft palette. Depending on intensity, snoring too may cause full or partial brain arousal during sleep.

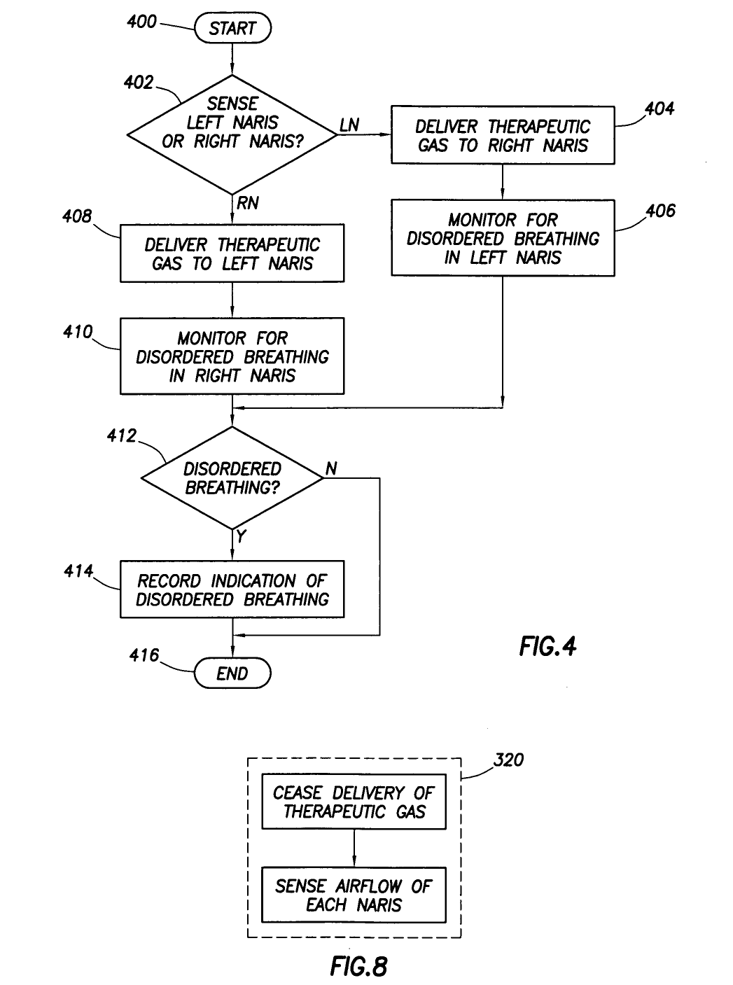

Thus, in situations wherein a particular breathing orifice is not a site for delivery of therapeutic gas, and the breathing orifice carries non-zero airflow, the monitoring and control system 10 monitors the patient for disordered breathing. FIG. 4 illustrates a method that may be implemented in accordance with at least some embodiments of the invention. The method of FIG. 4 may be incorporated with the illustrative method of FIG. 3, e.g., between start the timer (block 326 of FIG. 3) and expiration of the timer (block 328 of FIG. 3). Alternatively, the illustrative method of FIG. 4 could be implemented as a stand-alone process running substantially concurrently with FIG. 3. Further still, the illustrative method of FIG. 4 could operate alone, especially where a patient's only concern is a determination of the presence of disordered breathing while being provided a continues flow of therapeutic gas.

In particular, the illustrative method of FIG. 4 may start (block 400) and move to a determination of whether airflow should be sensed in the left or right naris (block 402). In embodiments operating in conjunction with the illustrative method of FIG. 3, the determination of where airflow should be sensed may be made by determining to which breathing orifice the monitoring and control system 10 is delivering therapeutic gas (blocks 318 and 322 of FIG. 3). In embodiments where the illustrative process of FIG. 4 operates standing alone, the determination may be made by the user interacting with the processor by way of a user interface 52 (FIG. 1). If the left naris (LN) is the site where sensing is to take place, the monitoring and control system 10 delivers therapeutic gas to the right naris (block 404) and monitors for disordered breathing in the left naris (block 406). If the method of FIG. 4 operates in conjunction with the illustrative method of FIG. 3, setting the monitoring and control system 10 to deliver therapeutic gas to the right naris and sense the left naris is completed by the steps of FIG. 3. If the illustrative method of FIG. 4 operates standing alone, then three-port valve 40 (FIG. 1) is commanded to couple the gas source 12 to the right naris, and three-port valve 42 (FIG. 2) is commanded to fluidly couple the flow sensor 48 to the left naris.

Still referring to FIG. 4, if the right naris (RN) is the site where sensing is to take place, the monitoring and control system 10 delivers therapeutic gas to the left naris (block 408) and monitors for disordered breathing in the right naris (block 410). If the method of FIG. 4 operates in conjunction with the illustrative method of FIG. 3, setting the monitoring and control system 10 to deliver therapeutic gas to the left naris and sense the right naris is completed by the steps of FIG. 3. If the illustrative method of FIG. 4 operates standing alone, three-port valve 42 (FIG. 1) is commanded to couple the gas source 12 to the left naris, and three-port valve 40 (FIG. 2) is commanded to fluidly couple the flow sensor 46 to the right naris.

Thereafter, a determination is made as to whether disordered breathing exists (block 412). In some embodiments, the Chicago criteria may be used to determine the presence of hypopnea. Apnea may be determined, for example, by sensing a reduction in measured breath volume (or other attribute proportional to volume) of 80% to 100% of non-hypopnea and/or non-apnea breathing, possibly in combination with time factor (e.g., 10 seconds) and/or drop in blood-oxygen saturation (e.g., falling below 90%). Snoring may be determined by sensing undulations in sensed airflow (or attribute proportional to airflow) having frequencies from 15 to 220 cycles per second. If disordered breathing is present (again block 412), an indication of the disordered breathing may be recorded (block 414) and the process ends (block 416), possibly by returning to the illustrative method of FIG. 3. If no disordered breathing is present, the process may end (block 416), again possibly by returning to the illustrative method of FIG. 3. Regardless of whether sleep disordered breathing is sensed in the right or left naris, the patient's continuous flow oxygen prescription may still be delivered during the sensing.

The embodiments discussed with respect to FIG. 4 operate by delivering a continuous flow of therapeutic gas to one naris, and sensing disordered breathing in a second naris. It would be advantageous, however, to deliver a continuous flow of therapeutic gas to each naris of a patient and simultaneously monitor for disordered breathing. FIG. 5 illustrates alternative embodiments that have the ability to deliver a continuous flow of therapeutic gas to each breathing orifice of a patient and simultaneously monitor for disordered breathing, in addition to the functionality discussed with respect to FIG. 1.

In particular, the monitoring and control system 10 of FIG. 5 comprises a processor 28, which may be a microcontroller (integral with ROM 30, RAM 32, D/A 34, A/D 36 and COM 38), or the system 10 of FIG. 5 may implement the functionality with stand-alone devices. The ROM 30 may comprise a software program that implements the various embodiments of the invention. Monitoring and delivery system 10 of FIG. 5 also comprises three valves 110, 112 and 114, each of which may be five-volt solenoid operated two port valve. By selectively applying voltage on a digital output signal line coupled to the valve 110, the processor 28 may be able to couple gas from the gas source 12 to the to the exemplary right naris. Valve 112, under command of the processor 28, may couple gas from the gas source 12 to exemplary left naris. Likewise, valve 114, under command of the processor 28, may couple gas from the gas source 12 to the patient's mouth.

Still referring to FIG. 5, a monitoring and control system 10 in accordance with these alternative embodiments may also comprise flow sensors 46, 48 and 50. Unlike the embodiments illustrated in FIG. 1, the flow sensors 46, 48 and 50 may fluidly couple to their respective breathing orifices at all times. Thus, monitoring and control system 10 may sense airflow associated with each breathing orifice at all times.

As illustrated in FIG. 5, the monitoring and control system 10 may couple to a patient by way of cannula 116. In these embodiments, cannula 116 may have six fluidly independent flow pathways to the patient 18, two each for each breathing orifice. In particular, the illustrative cannula 116 of FIG. 5 may comprise a first nasal tubing 150 that has a device end 152 configured to couple to the system 10 (such as by a Luer fitting) and an aperture end 154 configured to be in fluid communication with the left naris. The cannula may further comprise another nasal tubing 156 that has device end 158 configured to couple to the system 10 (such as by a Luer fitting) and an aperture end 160 configured to be in fluid communication with the right naris. The cannula may further comprise an oral nasal tubing 162 that has device end 164 configured to couple to the system 10 (such as by a Luer fitting) and an aperture end 166 configured to be in fluid communication with the mouth. The tubings 150, 156 and 162 may provide a mechanism to supply the therapeutic gas to the respective breathing orifice. The cannula 116 may further comprise another nasal tubing 168 that has device end 170 configured to couple to the system 10 (such as by a Luer fitting) and an aperture end 172 configured to be in fluid communication with the right naris. The cannula 116 may further comprise another nasal tubing 174 that has device end 176 configured to couple to the system 10 (such as by a Luer fitting) and an aperture end 178 configued to be in fluid communication with the right naris. Finally, cannula 116 may further comprise another nasal tubing 180 that has device end 182 configured to couple to the system 10 (such as by a Luer fitting) and an aperture end 184 configured to be in fluid communication with the right naris. The tubings 168, 174 and 180 may provide a mechanism to sense airflow, even when therapeutic gas is being delivered to the breathing orifice.

The cannula 116 may be constructed as an integral unit as illustrated, or may be implemented using two cannulas each having three fluidly independent pathways to the patient. Further still, the various fluidly independent pathways may be implemented with any of a combination of individual pieces of tubing, single lumen nasal cannulas and dual lumen nasal cannulas. In accordance with some embodiments, the tubing (individually or as part of one or more cannulas) fluidly coupled to the flow sensors 46, 48 and 50 may have a smaller diameter than the tubing (again individually or as part of one or more cannulas) through which therapeutic gas is provided to the patient, so as to reduce interference with the patient's breathing.

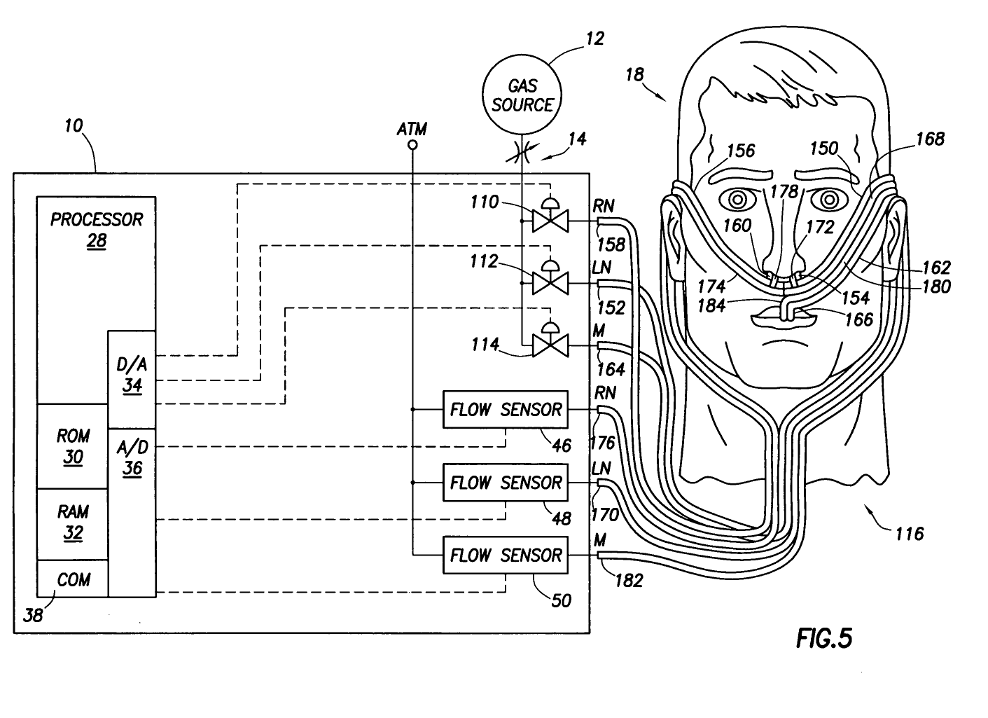

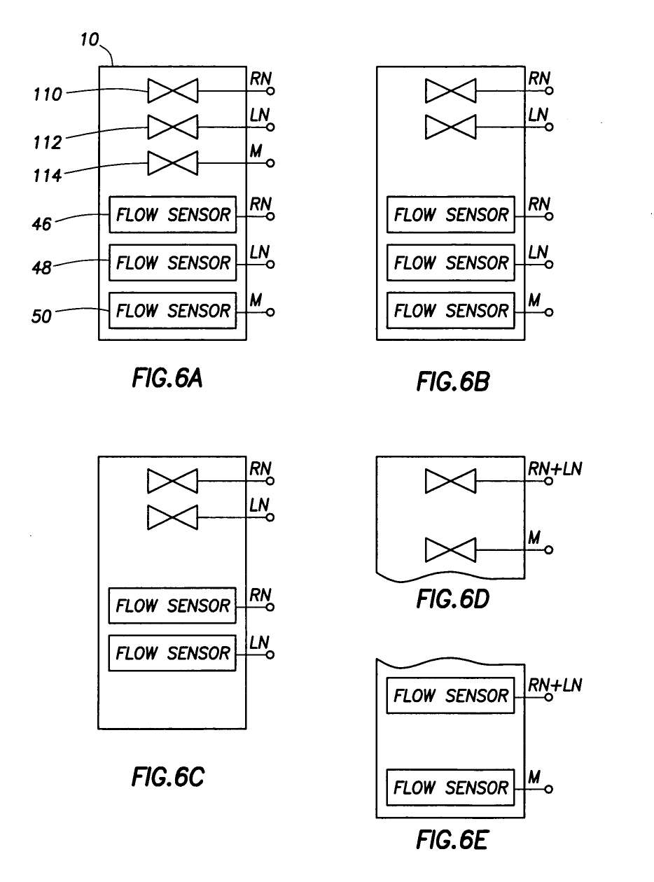

FIG. 6A illustrates the monitoring and control system 10 of FIG. 5 in a shorthand notation, showing only valves 110, 112 and 114 and flow sensors 46, 48 and 50. FIG. 2B illustrates alternative embodiments without the therapeutic gas flow pathway to the patient's mouth. In the embodiments illustrated in FIG. 6B, the therapeutic gas may fluidly couple to the patient by way of a bifurcated nasal cannula, and the flow sensors may couple to the patient by way of a cannula with three fluidly independent pathways. FIG. 6C illustrates alternative embodiments without the therapeutic gas flow pathway to the patient's mouth, and also without a flow sensor for the patient's mouth. In the embodiments illustrated in FIG. 6C, two bifurcated nasal cannulas may be used. FIG. 6D illustrates an alternative arrangement of the therapeutic gas flow valving where the nares are treated as a first group, and the mouth as a second group. Likewise, FIG. 6E illustrates an alternative arrangement of the flow sensors where the nares are treated as a first group, and the mouth as a second group. Moreover, the valving arrangement of FIG. 6D may be used with the flow sensor arrangement of FIG. 6E, or any of the flow sensor arrangements in FIGS. 6A-6C. Likewise, the flow sensor arrangement may be used with any of the valving arrangements of FIGS. 6A-6C. Finally, and though not specifically shown, any or all of the flow sensors in FIGS. 6A-6C and 6E may be equivalently replaced by pressure sensors (or other technology that senses attributes of respiratory airflow).

The embodiments of the monitoring and control system 10 of FIG. 5 may perform continuous flow therapeutic gas delivery to one breathing orifice while sensing airflow at a second breathing orifice as described with respect to the illustrative method of FIG. 3 and the system of FIG. 1. However, the embodiments of FIG. 5 also have the capability to sense airflow of a breathing orifice simultaneously with continuous flow delivery of therapeutic gas to that breathing orifice. This further means that the embodiments illustrated in FIG. 5 may also monitor for sleep disordered breathing at an orifice while supplying a continuous flow of therapeutic gas to the particular breathing orifice.

Consider a situation where the monitoring and control system 10 of FIG. 5 couples to the nares of the patient by way of a nasal cannula have four fluidly independent flow pathways to the nares (and with no fluid connection to the mouth of the patient for either sensing or delivery). Further consider that a patient's illustrative 2 LPM therapeutic gas flow prescription is being delivered through two of the four fluidly independent flow pathways. With reference to FIG. 5, this illustrative situation occurs when the flow control device 14 is set to allow a 2 LPM flow, and each of the valves 110 and 112 fluidly couple the gas source 12 to the patient. Assuming the resistance to gas flow through each flow pathway is approximately equal, this means each lumen or flow pathway of the therapeutic gas flow supply cannula carries approximately 1 LPM. The flow sensors 46 and 48 of FIG. 5 (and assuming flow sensor 50 is either not used or not present so as not to unduly complicate the discussion) thus sense airflow through each naris.

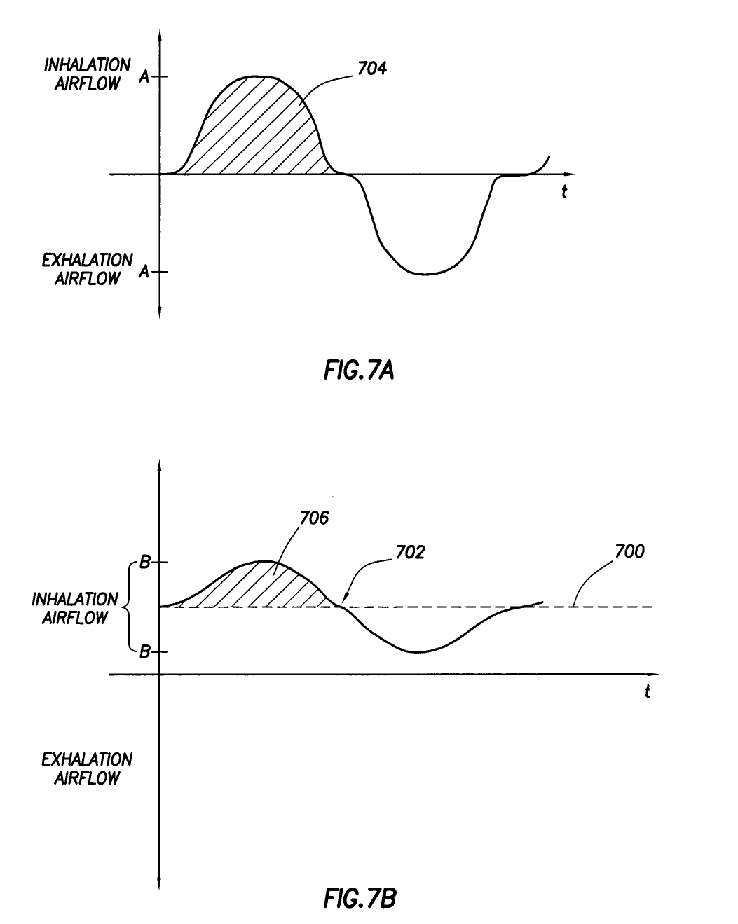

FIG. 7A illustrates a waveform of instantaneous measured airflow as a function of time for a breathing orifice coupled to a flow sensor by way of a tube (e.g., of a nasal cannula) when there is no attempt to simultaneous delivery therapeutic gas while measuring. Such a waveform is centered at a no-flow, has illustrative inhalation and exhalation mode amplitude “A”, and thus has a peak-to-peak amplitude of 2A. FIG. 7B illustrates a waveform of instantaneous measured airflow as a function of time for a breathing orifice coupled to a flow sensor by way of tube (e.g., of a nasal cannula) where the sensing takes place simultaneously with a continuous flow delivery of therapeutic gas to that breathing orifice. The inventors of the present specification have found that continuous flow delivery of therapeutic gas affects sensed airflow in at least two ways. Given that the aperture end or nasal prong of a cannula sensing airflow will be proximate to a nasal prong of a cannula delivering a continuous flow delivery of therapeutic gas. The continuous flow delivery of therapeutic gas creates a moving air stream, which air stream draws air through the tubing of the nasal cannula which thus appears to be an inhalation. However, the apparent inhalation is constant throughout the breathing cycle, and thus appears as a bias, or inhalation bias, in the measured airflow waveform. In FIG. 7B, this bias is illustrated by dashed line 700. Thus, even when the patient is neither inhaling nor exhaling, for example at point 702, there still exists a measured airflow. When determining the relative inhalation volumes of each breathing orifice, this bias in airflow may be taken into account. In spite of the bias in airflow, however, the patient's respiratory airflow waveform may still be visible in the sensed airflow.

Still referring to FIG. 7B, the second manifestation of sensing respiratory airflow simultaneously with a continuous flow delivery of therapeutic gas is in terms of amplitude of the sensed waveform. In particular, the higher the therapeutic gas flow rate provided to the patient, the greater the dampening effect on the sensed airflow. Thus, FIG. 7B illustrates an amplitude B (from the bias line 700), and thus the peak-to-peak amplitude of the illustrative waveform of FIG. 7B is 2B. If an actual respiratory airflow is the same in each case, the peak-to-peak amplitude when measuring respiratory airflow without simultaneous delivery of therapeutic gas will be greater than the peak-to-peak amplitude where sensing takes place simultaneously with delivering of therapeutic gas (FIG. 7B).

Thus, in embodiments where respiratory airflow is sensed simultaneous with continuous flow delivery of therapeutic gas, volume calculations may take into account the inhalation bias associated with the continuous flow delivery. For example, and referring to FIG. 7A, the inhalation volume for the exemplary waveform would be the area 704 under the inhalation curve (cross-hatched). Likewise with respect to FIG. 7B, the volume of the inhalation waveform 706 would be the area under the inhalation waveform up to the line defining the inhalation bias caused by the continuous flow of therapeutic gas. In embodiments of the monitoring and control system 10 of FIG. 5 that also monitor for sleep disorder breathing, apnea and hypopnea determinations may be made based on volume 706 and/or amplitude of the inhalation waveform (from the bias line 700). With respect to snoring, the snoring waveform may “ride” the inhalation waveform and the effect of the continuous flow of therapeutic gas is to dampen the peak-to-peak amplitude of the snoring signal as it “rides” the inhalation waveform. However, the snoring waveform, in its analog or electronic form, may be separated from the inhalation waveform by applying the combined waveform to a either a hardware or software high pass filter respectively. Although the illustrative embodiments of FIG. 7B show the inhalation bias moving the exemplary waveform completely above the zero flow line, the amount of inhalation bias is proportional to the patient's continuous flow therapeutic gas delivery prescription. Thus, the exemplary waveform may have an inhalation bias such that the entire waveform is above the no-flow condition, but also the exhalation portion of the waveform may extend below the actual no-flow line, meaning that the patient's exhalation may overcome any inhalation bias thus reversing airflow through the cannula or other sensing tube.

In all of the embodiments, in the event an inhalation is not detected through any breathing orifice, an alarm may be sounded. Relatedly, an apnea event is sensed, an alarm may be sounded. Moreover, the patient's breathing patterns may be stored, such as in RAM 16, and communicated to external devices through communication port 17.

The various embodiments discussed to this point have been described as delivering a continuous flow of therapeutic gas while measuring airflow. In alternative embodiments, the continuous flow of therapeutic gas may cease for a predetermined number of respirations (e.g., a single respiratory cycle, or multiple respirator cycles) while the airflow through each breathing orifice is measured. Based on measured airflow, continuous flow of therapeutic gas may be provided substantially only to the breathing orifice(s) exhibiting the greater air flow, or to each breathing orifice. Referring again briefly to FIG. 3, in these alterative embodiments, the illustrative delivering therapeutic gas to the left naris and sensing airflow of the right naris (block 302) and the illustrative delivering therapeutic gas to the right naris and sensing airflow of the left naris (block 304) would be combined and modified to be ceasing delivery of therapeutic gas and sensing airflow of each naris (block 330), as illustrated in FIG. 8. The remaining portions of FIG. 3 could remain unchanged in these alternative embodiments.

The above discussion is meant to be illustrative of the principles and various embodiments of the present invention. Numerous variations and modifications are possible. For example, in embodiments where the monitoring and control system 10 is to be a portable, battery operated device, latching values may be used to reduce battery usage. Moreover, during the period of time when the monitoring and control system 10 is delivering therapeutic gas to the one or more selected breathing orifices, non-vital components may be powered down to conserve battery power. It is intended that the following claims be interpreted to embrace all such variations and modifications.

What is claimed is:

1. A method comprising:
sensing an attribute of respiratory airflow of a first breathing orifice of a patient; and
delivering a continuous flow of therapeutic gas to a second breathing orifice of the patient simultaneously with the sensing.

2. The method as defined in claim 1 further comprising, after the sensing and delivering:
sensing an attribute of respiratory airflow of the second breathing orifice; and
delivering a continuous flow of therapeutic gas to the first breathing orifice simultaneously with sensing the attribute of respiratory airflow of the second breathing orifice.

3. The method as defined in claim 2 further comprising, after sensing and delivering, delivering a continuous flow of therapeutic gas to one of the first breathing orifice or second breathing orifice carrying greater air flow.

4. The method as defined in claim 3 further comprising, after sensing and delivering, delivering a continuous flow of therapeutic gas to each of the first breathing orifice and second breathing orifice.

5. The method as defined in claim 3 wherein the first breathing orifice is a first naris of the patient, and the second breathing orifice is a second naris of the patient.

6. The method as defined in claim 3 wherein the first breathing orifice is a nose of the patient, and the second breathing orifice is a mouth of the patient.

7. The method as defined in claim 2 wherein sensing further comprises sensing at least a portion of the airflow of the breathing orifice.

8. The method as defined in claim 2 further comprising:
calculating a total breath volume based on the sensing; and
delivering the continuous flow of therapeutic gas to the first breathing orifice if a breath volume carried by the first breathing orifice is greater than a predetermined threshold.

9. The method as defined in claim 8 wherein the predetermined threshold is 75% of the total breath volume.

10. The method as defined in claim 8 further comprising delivering a continuous flow of therapeutic gas to each of the first breathing orifice and second breathing orifice if a breath volume carried by each of the first breathing orifice and second breathing orifice is within a predetermined threshold.

11. The method as defined in claim 10 wherein the predetermined threshold is between 25% and 75% of the total breath volume.

12. The method as defined in claim 1 wherein sensing further comprises checking for the presence of at least one of hypopnea, apnea or snoring.

13. The method as defined in claim 1 further comprising, simultaneous with sensing and delivering:
sensing an attribute of respiratory airflow of the second breathing orifice; and
delivering a continuous flow of therapeutic gas to the first breathing orifice simultaneously with the sensing the attribute of respiratory airflow of the second breathing orifice.

14. The method as defined in claim 13 wherein sensing further comprises checking for the presence of at least one of hypopnea, apnea or snoring.

15. A method comprising:
delivering therapeutic gas to one or more breathing orifices of a patient; and then
ceasing delivery of therapeutic gas for a predetermined number of respirations and sensing an attribute of airflow through each breathing orifice during the ceasing; and thereafter
delivering a continuous flow of therapeutic gas one of: substantially only to the breathing orifice exhibiting greater airflow; or to each breathing orifice.

16. The method as defined in claim 15 wherein ceasing and sensing further comprises ceasing delivery of therapeutic gas for a single respiratory cycle.

17. The method as defined in claim 15 further comprising, simultaneously with the sensing, checking for the presence of at least one of hypopnea, apnea or snoring.

18. The method as defined in claim 15 wherein delivering the continuous flow further comprises delivering the continuous flow of therapeutic gas one of: to substantially only a first naris of the patient; or to each naris of the patient.

19. the method as defined in claim 15 wherein delivering the continuous flow further comprises delivering the continuous flow of therapeutic gas one of: substantially only to the nose of the patient; or to each of the nose and mouth of the patient.

20. A system comprising:
a processor;
a first sensor electrically coupled to the processor and configured to fluidly couple to a breathing orifice of a patient, the sensor senses an attribute of airflow of the breathing orifice; and
a first valve electrically coupled to the processor and configured to selectively fluidly couple a source of therapeutic gas to a breathing orifice;
wherein the system is configured to sense the attribute of airflow of a first breathing orifice, sense the attribute of airflow of a second breathing orifice, and based at least in part on the attributes of airflow sensed, one of: deliver a continuous flow of therapeutic gas to only the first breathing orifice; deliver the continuous flow of therapeutic gas to only a second breathing orifice; or deliver the continuous flow of therapeutic gas to each of the first and second breathing orifices.

21. The system as defined in claim 20 wherein the system is configured to simultaneously sense the attribute of airflow of the first breathing orifice and deliver the continuous flow of therapeutic gas to the second breathing orifice.

22. The system as defined in claim 21 wherein the system is configured to simultaneously sense the attribute of airflow of the second breathing orifice and deliver the continuous flow of therapeutic gas to first breathing orifice

23. The system as defined in claim 22 wherein the system is configured to sense the attribute of the first breathing orifice and deliver to the second breathing orifice, and thereafter senses the attribute of the second breathing orifice and delivers to the second breathing orifice.

24. The system as defined in claim 22 wherein the system is configured to sense the attribute of the first breathing orifice and deliver to the second breathing orifice, and simultaneously sense the attribute of the second breathing orifice and deliver to the second breathing orifice.

25. The system as defined in claim 20 further comprising:
wherein the first sensor is configured to fluidly couple to the first breathing orifice;
wherein the first valve is configured to selectively fluidly couple the source of therapeutic gas to the first breathing orifice;
a second sensor electrically coupled to the processor and configured to fluidly couple to the second breathing orifice, the sensor senses an attribute of airflow of the second breathing orifice;
a second valve electrically coupled to the processor and configured to selectively couple the source of therapeutic gas to the second breathing orifice;
wherein the system is configured to sense the attribute of airflow of the first breathing orifice with the first sensor and simultaneously deliver the continuous flow of therapeutic gas to the second breathing orifice with the second valve, and thereafter to sense the attribute of airflow of the second breathing orifice with the second sensor and simultaneously deliver the continuous flow of therapeutic gas to the first breathing orifice with the first valve.

26. The system as defined in claim 20 further comprising
wherein the first sensor is configured to fluidly couple to the first breathing orifice;
wherein the first valve is configured to selectively fluidly couple the source of therapeutic gas to the first breathing orifice;
a second sensor electrically coupled to the processor and configured to fluidly couple to the second breathing orifice, the sensor senses an attribute of airflow of the second breathing orifice;
a second valve electrically coupled to the processor and configured to selectively couple the source of therapeutic gas to the second breathing orifice;
wherein the system is configured to sense the attribute of airflow of both the first and second breathing orifices and simultaneously deliver the continuous flow of therapeutic gas to both the first and second breathing orifices.

27. The system as defined in claim 20 wherein the system is configured to cease delivery of the therapeutic gas, and while the therapeutic gas delivered is ceased the system is configured to sense the attributes of airflow.

28. The system as defined in claim 20 wherein, during the period of time when the system senses the attribute of airflow, the system is further configured to check for the presence of at least one of hypopnea, apnea or snoring.

29. A cannula comprising:
a first nasal tubing having a device end and an aperture end, wherein the cannula is configured to place the aperture end in fluid communication with a first naris of a patient;
a second nasal tubing having a device end and an aperture end, wherein the cannula is configured to place the aperture end of the second nasal tubing in fluid communication with a second naris of the patient; and
an oral tubing having a device end and a first and second aperture ends, and the oral tubing mechanically coupled to at least one of the first or second nasal tubing, wherein the cannula is configured to place the aperture ends of the oral tubing in fluid communication with a mouth of the patient;
wherein the first nasal tubing, the second nasal tubing and the oral tubing are fluidly independent between their aperture ends and their device ends.

30. The cannula as defined in claim 29 wherein the oral tubing further comprises:
a first oral tubing having the device end and the first aperture end; and
a second oral tubing section having a device end and the second aperture end;
wherein the first and second oral tubings are fluidly independent between their aperture ends and their device ends.

31. The cannula as defined in claim 29 wherein the oral tubing is coupled parallel along at least a part of the first nasal tubing.
